(19) United States
(12) Patent Application Publication
(10) Pub. No.: US 2020/0297441 A1
(43) Pub. Date: Sep. 24, 2020

(54) SURGICAL DEVICES AND METHODS FOR COLLECTING STERILIZATION DATA

(71) Applicant: Covidien LP, Mansfield, MA (US)

(72) Inventor: Richard Lech, Hamden, CT (US)

(21) Appl. No.: 16/769,780

(22) PCT Filed: Dec. 19, 2018
(86) PCT No.: PCT/US2018/066420

§ 371 Date: Jun. 4, 2020

Related U.S. Application Data
(60) Provisional application No. 62/613,457, filed on Jan. 4, 2018.

(51) Int. Cl. A61B 34/35; A61B 34/37; A61B 90/00; A61B 90/70

(52) U.S. Cl. CPC A61B 34/35; A61B 34/37; A61B 90/06; A61B 2034/302; A61B 90/70; A61B 2562/0271; A61B 90/08

(57) ABSTRACT

A surgical device is provided including an energy harvesting device configured to harvest energy from a sterilization device to provide power to at least one of a sensor or a computing device. The sensor is configured to measure an energy output applied to the surgical device by a sterilization device. The computing device includes a processor or logic circuit and a memory and is configured to determine when a threshold energy output applied to the surgical device is met and to store in the memory an amount of times the threshold energy output applied to the surgical device is met.

### BACKGROUND

Robotically-assisted surgery is increasingly being used in minimally invasive medical procedures. Some robotic surgical systems include a console supporting a surgical robotic arm and a robotic surgical instrument mounted to the robotic arm. The robotic surgical instrument may have an elongated shaft that supports at least one end effector (e.g., forceps or a grasping tool) on a distal end thereof.

After each use, the robotic surgical instrument is disposed of, reused, or partially disposed of and partially reused. Any part of a surgical instrument that is reused must be cleaned or sterilized to neutralize potentially infectious agents before being reused. Sterilization devices (e.g., pressure chambers, autoclaves, etc., or combinations thereof) are used to sterilize reusable surgical instruments. Typically, the surgical instrument is placed in the sterilization device for a period of time during which it is exposed to some form of energy (e.g., heat, light, kinetic, pressure, etc., or combinations thereof) in order to remove the infectious agents from the surgical instrument. However, repeated exposure of the surgical instrument to the sterilization device can degrade or deteriorate electrical and mechanical components within the housing of the surgical instrument. In many situations, it is difficult to determine how many times a particular surgical instrument has been sterilized.

Accordingly, a need exists for a surgical instrument capable of determining and recording sterilization data during a sterilization procedure.

### SUMMARY

The present disclosure relates to surgical devices configured to be powered by and operate during a sterilization procedure to record sterilization data.

According to an aspect of the present disclosure, a surgical device is provided, including an energy harvesting device configured to harvest energy from a sterilization device to provide power to at least one of a sensor and a computing device. The sensor is configured to measure an energy output applied to the surgical device by a sterilization device. The computing device includes a processor and/or logic circuit and a non-volatile memory and may be configured to determine when a threshold energy output applied to the surgical device is met and to store in the memory an amount of times the threshold energy output applied to the surgical device is met.

In embodiments, when the threshold energy output applied to the surgical device is met, the computing device counts and the memory records a completed sterilization cycle.

In some embodiments, the computing device may be configured to determine when the surgical device requires servicing based on a number of completed sterilization cycles.

In certain embodiments, the energy harvesting device may be a thermoelectric generator configured to receive a concentration of thermal energy from a sterilization device. The thermoelectric generator may be configured to convert a concentration of thermal energy into electrical energy to power the surgical device.

In embodiments, the sensor may be a temperature sensor configured to measure a temperature of an outer surface or the differential temperature between the inner and outer surfaces of the surgical device.

In some embodiments, the temperature sensor may be operatively coupled to the computing device. The computing device may be configured to detect a threshold temperature necessary to sterilize the surgical device.

In certain embodiments, when the computing device detects a threshold temperature, the computing device counts and the memory records a completed sterilization cycle.

In embodiments, the surgical device may include a display configured for displaying at least one output of the computing device.

In some embodiments, the surgical device may include an energy storage device configured to store the harvested energy from the energy harvesting device.

In certain embodiments, the energy storage device may be a capacitor.

In embodiments, the surgical device may include an amplifier operatively connected to the energy harvesting device. The amplifier may be configured to condition the power (e.g., to increase the output voltage) of the harvested energy from the energy harvesting device.

In some embodiments, the energy harvesting device may include a photovoltaic cell configured to convert light energy from a sterilization device into electrical energy to power the surgical device.

In certain embodiments, the energy harvesting device may include a piezoelectric transducer configured to convert kinetic energy into electrical energy to power the surgical device.

In embodiments, the surgical device may include a data interface device configured to interface with an external device. The data interface device may be configured to upload data from the computing device onto the external device and to download data from the external device onto the computing device.

According to another aspect of the present disclosure, a method for operation of a surgical instrument during sterilization is provided, including placing a surgical instrument in a sterilization device. The surgical instrument includes an energy harvesting device configured to harvest energy from a sterilization device, a sensor configured to measure an energy output applied to the surgical device by a sterilization device, and a computing device including a processor and/or logic circuit, and a memory. The method also includes powering the sensor and the computing device using the harvested energy from the energy harvesting device, determining, with the computing device and the sensor, when a threshold energy output applied to the surgical device is met, and storing in the memory an amount of times that the threshold energy output applied to the surgical device is met.

In embodiments, the method may include counting, with the computing device, a completed sterilization cycle when a threshold energy output is applied to the surgical device.

In some embodiments, the method may include disabling the surgical device with the computing device when a threshold number of completed sterilization cycles is met.

In certain embodiments, the method may include storing the harvested energy from the energy harvesting device with an energy storage device.

In embodiments, the method may include displaying on a display device at least one output of the computing device.

In some embodiments, the method may include conditioning the harvested energy from the energy harvesting device with an amplifier.

### BRIEF DESCRIPTION OF THE DRAWINGS

Embodiments of the present surgical devices are described herein with reference to the accompanying drawings, wherein:

FIG. 1 is a schematic illustration of a robotic surgical system in accordance with the present disclosure;

FIG. 2 is a perspective view of a surgical assembly of the robotic surgical system of FIG. 1;

FIG. 3 is a perspective view of a surgical instrument of the surgical assembly of FIG. 2;

FIG. 4 is a schematic diagram of a circuit board of the surgical instrument of FIGS. 2 and 3; and

FIG. 5 is a flowchart depicting operation of the surgical instrument of FIGS. 2 and 3 during a sterilization procedure.

### DETAILED DESCRIPTION

Embodiments of the present surgical devices will now be described in detail with reference to the drawings, in which like reference numerals designate identical or corresponding elements in each of the several views. As used herein, the term “clinician” refers to a doctor, nurse, or other care provider and may include support personnel. In the following description, well-known functions or constructions are not described in detail to avoid obscuring the present disclosure in unnecessary detail. As used herein, the term “distal,” as is conventional, will refer to that portion of the instrument, apparatus, device or component thereof which is closer to farther the patient while, the term “proximal,” will refer to that portion of the instrument, apparatus, device or component thereof which is further from the patient.

The present disclosure relates to surgical instruments. Specifically, the present disclosure is directed to a surgical instrument configured to harvest energy from a sterilization device. The harvested energy from the sterilization device is used to power the surgical instrument while the surgical instrument is in the sterilization device to collect sterilization data. The surgical instrument then stores the sterilization data in memory. The stored sterilization data can be used to notify a user if the surgical instrument and/or parts thereof are fit for continued use, need replacement, and/or should be discarded or completely disable the instrument when all uses have expired.

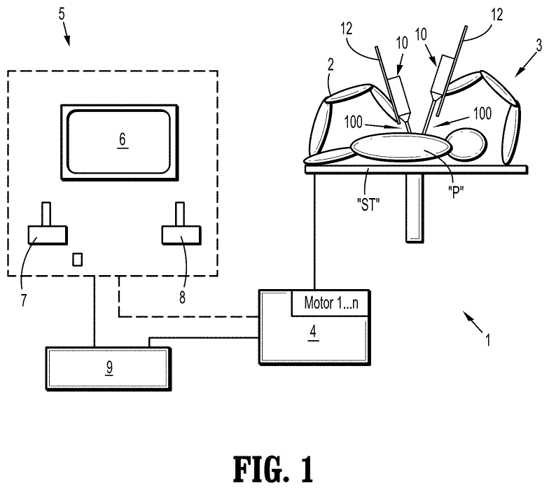

Referring initially to FIG. 1, a robotic surgical system, such as, for example, medical work station 1, generally includes a plurality of robot arms 2 and 3, a control device 4, and an operating console 5 coupled with control device 4. Operating console 5 includes a display device 6 configured to display three-dimensional images, and manual input devices 7 and 8, a clinician can use to telemanipulate (not shown) robot arms 2 and 3 in a first operating mode, as known in principle to a person skilled in the art.

Each of the robot arms 2 and 3 includes a plurality of members, which are connected through joints, and which may be releasably attached to a surgical assembly 10. Robot arms 2 and 3 may be driven by electric drives (not shown) that are connected to control device 4. Control device 4 (e.g., a computer) is set up to activate the drives (e.g., using a computer program) and execute a desired movement of robot arms 2 and 3 and/or surgical assembly 10 according to a movement defined by means of manual input devices 7 and 8. Control device 4 may be configured to regulate the movement of robot arms 2 and 3 and/or of the drives (not shown). Control device 4 may control a plurality of motors, e.g., “Motor 1 . . . n,” with each motor configured to drive movement of robotic arms 2 and 3 in a plurality of directions.

Medical work station 1 is configured for use on a patient “P” lying on a patient table “ST” to be treated in a minimally invasive manner by or with surgical instrument 100 of surgical assembly 10. Medical work station 1 may include more than two robot arms 2 and 3, the additional robot arms likewise being connected to control device 4 and being telemanipulatable by means of operating console 5. A surgical assembly 10 may be attached to the additional robot arm. Medical work station 1 may include a database 9 operatively (e.g., directly and/or indirectly) coupled with control device 4. Database 9 may store, e.g., pre-operative data from patient “P” and/or anatomical atlases.

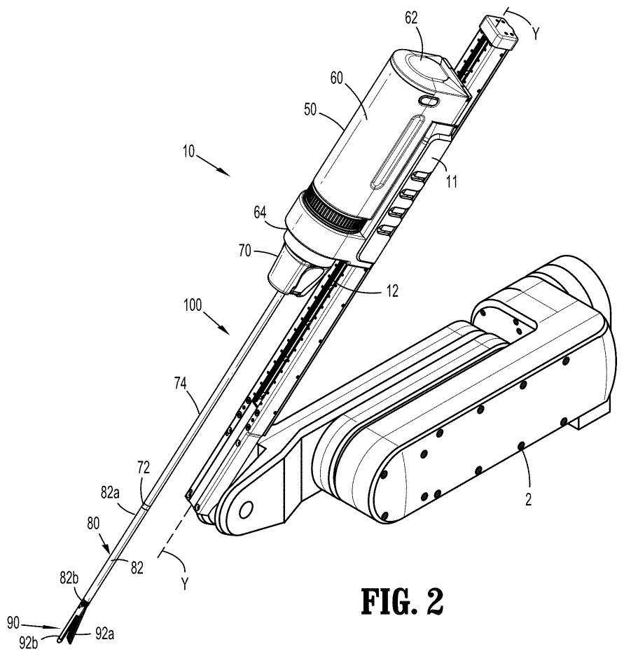

Turning now to FIG. 2, surgical assembly 10 is shown coupled with or to robotic arm 2 via a rail, track, or slide 12. While surgical assembly 10 is discussed singularly, a person of ordinary skill in the art can readily appreciate that the medical work station 1 may also include a plurality of substantially identical surgical assemblies 10 coupled with or to each of the robotic arms 2 and 3 (FIG. 1). Surgical assembly 10 includes an instrument drive unit 50 coupled to an adapter or instrument drive connector 70 of surgical instrument 100 having a surgical loading unit 80 including an end effector 90 disposed at a distal end thereof.

Instrument drive unit 50 of surgical assembly 10 may be supported on or connected to a slider 11 that is movably connected to a track 12 of robotic arm 2. Slider 11 moves, slides, or translates along a longitudinal axis “Y” defined by track 12 of surgical robotic arm 2 upon a selective actuation by motors (not shown) disposed in track 12 of robotic arm 2 or motors (e.g., one or more of “Motor 1 . . . n”) of control device 4. As such, slider 11, with surgical assembly 10 connected thereto, can be moved to a selected position along track 12 of robotic arm 2.

Instrument drive unit 50 includes a housing 60 having a proximal end 62 and a distal end 64 configured to be operably coupled to instrument drive connector 70 of surgical instrument 100. Housing 60 of instrument drive unit 50 houses a plurality of motors (not shown) that are configured to power surgical instrument 100, for example, to drive various operations of end effector 90 of surgical instrument 100. Thus, in use, instrument drive unit 50 transfers power and actuation forces from the motors to instrument drive connector 70 of surgical instrument 100 to drive movement of end effector 90 of surgical instrument 100.

Control device 4 (FIG. 1) may control the motors of instrument drive unit 50. In some embodiments, one or more motors may receive signals wirelessly (e.g., from control device 4). It is contemplated that control device 4 coordinates the activation of the various motors (“Motor 1 . . . n”), and the motors of instrument drive unit 50, to coordinate an operation and/or movement of surgical instrument 100.

Surgical loading unit 80 is selectively attachable to instrument drive connector 70 and includes an elongate portion 82 and an end effector 90. Surgical loading unit 80 may be a single use loading unit that is disposable, or a multiple use loading unit that can be sterilized in a sterilization device for reuse. Elongate portion 82 of surgical loading unit 80 may have a proximal end 82a configured to be coupled to a distal cap 72 of an elongated shaft 74 of instrument drive connector 70. Elongate portion 82 of surgical loading unit 80 has a distal end 82b having end effector 90 attached thereto. End effector 90 generally includes a pair of opposing jaw members 92a and 92b, and may include a staple cartridge, knife blade, among other fastening, cutting, clamping elements within the purview of those skilled in the art. It is contemplated that end effector 90 may be directly coupled to instrument drive connector 70 rather than be directly coupled to elongate portion 82 of surgical loading unit 80.

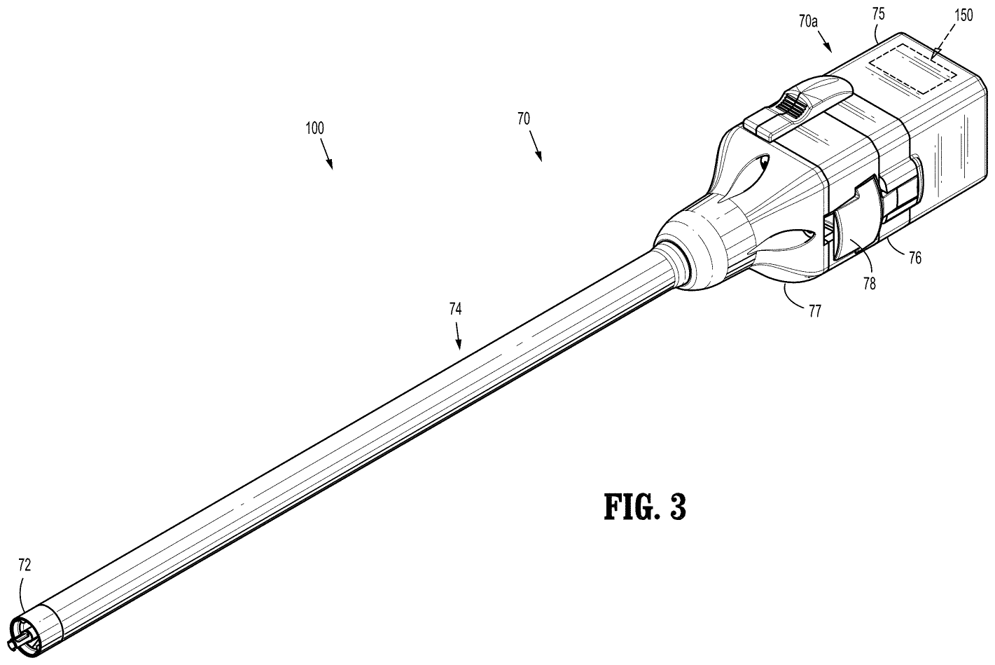

Referring now to FIG. 3, instrument drive connector 70 of surgical instrument 100 includes a housing assembly 70a and elongated shaft 74 extending distally therefrom and terminating at distal cap 72. Housing assembly 70a includes a proximal housing 75, a bottom or distal housing 76, a tip housing 77, and a circuit board 150 disposed within housing assembly 70a for controlling various operations of surgical instrument 100, as will be described in detail below.

For a detailed discussion of the construction and operation of a similar robotic surgical system having one or more of the same or similar components for use with one or more components of the presently described robotic surgical system, reference may be made to U.S. Pat. No. 8,828,023, the entire disclosure of which is incorporated by reference herein.

Reference may be made to commonly owned International Patent Application No. PCT/US14/61329, U.S. Pat. No. 8,636,192, or U.S. Pat. No. 8,925,786, the entire disclosures of each of which are incorporated by reference herein, for a detailed discussion of illustrative examples of the construction and operation of end effectors for use with, or connection to, the presently disclosed electromechanical surgical instruments.

As can be appreciated, surgical instruments are often reused from one procedure to the next. Any part of a surgical instrument that is reused must be sterilized to neutralize potentially infectious agents before being reused. Sterilization devices, e.g., pressure chambers, autoclaves, etc., or combinations thereof, are used in medical applications to sterilize surgical instruments. However, the repeated exposure to elevated temperatures, pressure, or other forms of energy emitted from the sterilization device may cause a surgical instrument or components thereof to malfunction or become inoperable. Thus, the usage of a surgical instrument may be limited by the number of times that the surgical instrument has been placed in a sterilization device for sterilization.

The surgical instrument 100 of the present disclosure is configured to provide a user with sterilization cycle data without a need for the user to independently keep track of sterilization data or to use external devices to keep track of sterilization data.

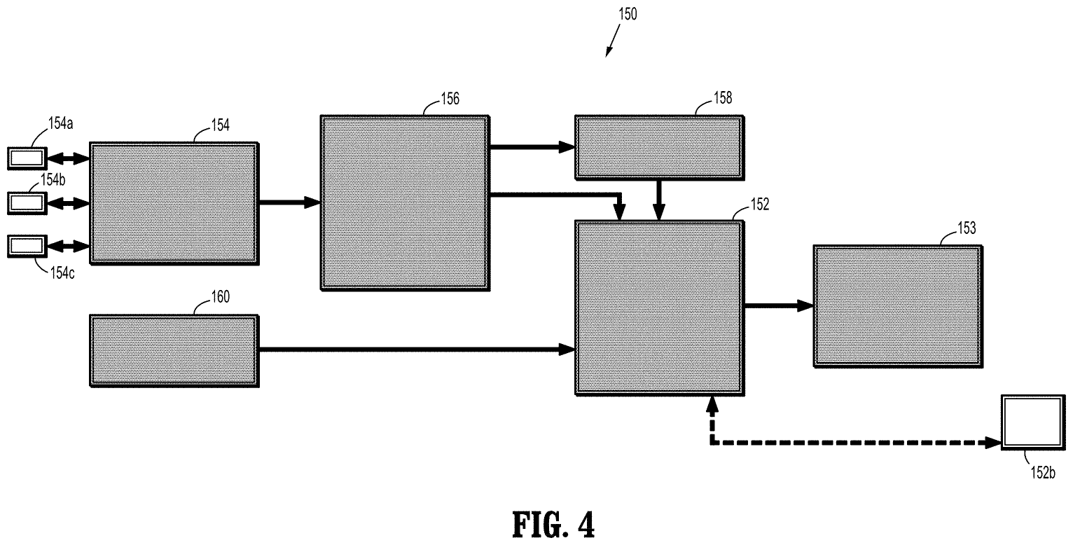

With reference to FIGS. 3 and 4, in an embodiment, for example, for energy harvesting, circuit board 150 of surgical instrument 100 generally includes a device configured to implement (or capable of implanting) a state machine such as a central processing unit (CPU) or logic circuit (hereinafter, “CPU”) 152, a display 153 (optionally), an energy harvesting device 154 configured to provide power to CPU 152, an amplifier 156 configured for conditioning the energy generated by the energy harvesting device 154, an energy storage device 158 (optional) configured to store the harvested energy from the energy harvesting device 154, and a sensor 160 for measuring the energy output of the sterilization device, as applied to the surgical instrument 100.

In general, CPU 152 of circuit board 150 is configured to operate in conjunction with other components of surgical instrument 100 as described herein, to calculate and/or determine sterilization data, e.g., the number of times that that surgical instrument 100 has undergone sterilization in a sterilization device. Based on such sterilization data, CPU 152 is configured to determine whether surgical instrument 100, and/or any parts/components thereof, need to be serviced, replaced, and/or discarded. CPU 152 may also be configured to perform a “self-destruct” operation to render surgical instrument 100, and/or components thereof permanently inoperable, or unusable without voluntary user intervention. Specifically, if surgical instrument 100 has undergone a threshold limit or number of sterilization cycles, CPU 152 will automatically prohibit further use of surgical instrument 100, or parts thereof, until certain components of surgical instrument 100 are replaced, serviced, disabled, or discarded. The threshold limit of the number of times that surgical instrument 100 or any part thereof can undergo a sterilization cycle may be pre-programmed into CPU 152, or based on empirical or experimental data.

For example, after a threshold number of sterilization cycles have been performed on surgical instrument 100, CPU 152 will indicate that a particular component of surgical instrument 100 requires servicing or replacement, or that surgical instrument 100 must be discarded. Specifically, CPU 152 may determine that surgical instrument 100 is no longer safely usable after undergoing a threshold number of sterilization cycles. CPU 152 will engage an auto lock to prevent surgical instrument 100 from being used. In embodiments, CPU 152 may be configured to determine a failure of surgical instrument 100 or any part thereof before the threshold limit of sterilization cycles is met.

In embodiments, CPU 152 of circuit board 150 may be any type of suitable processor or computer adapted to perform or execute techniques, operations, and/or instructions described herein. For example, the CPU 152 may be hardware processors programmed to perform the techniques described herein pursuant to the instructions in firmware, memory, or other storage, or a combination thereof. Similarly, CPU 152 may be one or more application specific integrated circuits (ASICs), field programmable gate arrays (FPGAs), Complex Programmable Logic Devices (CPLD) that are persistently programed to perform the techniques or operations described herein. CPU 152 may also be a digital signal process (DSP), a microprocessor, microcontroller, or any other device that incorporates hard wired logic or program logic or both to perform the operations or techniques described herein.

CPU 152 of circuit board 150 includes memory 152a which may be any type of hardware device used to store information from CPU 152 (e.g., sterilization data and service information), such as random access memory (RAM). The memory 152a may be non-volatile memory, such as read-only memory (ROM) (e.g., programmable read-only memory (EPROM), electrically erasable programmable read-only memory (EEPROM), and/or non-volatile RAM (NVRAM), etc., or combinations thereof). The memory may also be magnetic, optical, or electrical media.

A digital display 153 of surgical instrument 100 may be operatively (e.g., directly and/or indirectly) connected to CPU 152. Digital display 153 may be any device configured to display at least one output of CPU 152, e.g., sterilization data, service information, or an indication of a condition, as calculated and/or determined by CPU 152. For example, digital display 153 may provide a user with a numerical representation of successfully performed sterilization cycles on surgical instrument 100. Digital display 153 may provide a user with service information, such as indicating an imminent need to replace, service, or dispose of one or more parts/components of surgical instrument 100, e.g., “Replace Part X,” “Service Part Y,” “Discard Part Z,” Discard Instrument B,” or the like.

The display 153 may be a liquid crystal display, a plasma display, one or more light emitting diodes, a luminescent display, a multi-color display, an analog display, a passive display, an active display, a “twisted nematic” display, a “super twisted nematic” display, a “dual scan” display, a reflective display, a backlit display, an alpha numeric display, a monochrome display, a “Low Temperature Polysilicon Thin Film Transistor” or LPTS TFT display, Organic LED (OLED) Display, machine-encapsulated electrophoretic display such as an E-Ink (electronic ink) Di splay (a microencapsulated electrophoretic display), or any other display 153 that indicates a parameter, information, or graphics related to service or sterilization data of the surgical instrument 100. In certain embodiments, display 153 may include a mechanical indicator that is configured to provide any suitable audible, tactile, and/or visual output.

Surgical instrument 100 may include a data interface device 152b configured to interface with CPU 152 and/or an external device, e.g., a tablet, smart device, computer, etc., or combinations thereof. Data interface device 152b may be configured to download data from CPU 152 and/or memory 152a onto an external device. Additionally, data interface device 152b may be configured to upload data from an external device onto CPU 152 and/or memory 152a to change and/or update the programming of CPU 152 (e.g., firmware updates). Data interface device 152b may be a Universal Serial Bus (USB) connector configured to interface with an external device using a USB cable. Additionally or alternatively, data interface device 152b may be configured to receive a USB flash media drive, an SD card, or other removable or non-removable storage medium to download/upload data from CPU 152 and/or memory 152a. Data interface device 152b may also include, and is not limited to, Ethernet, Serial Peripheral Interface (SPI), Inter-Integrated Circuit (I2C), 1-Wire, or any other proprietary interface.

Energy harvesting device 154 may be any suitable device configured for deriving energy (e.g., light, thermal, kinetic, RF, pressure, etc., or combinations thereof) from a sterilization device (e.g., an autoclave) and using the energy from the sterilization device to activate or “power on” CPU 152 such that surgical instrument 100 can collect sterilization data during a sterilization procedure. Energy harvesting device 154 provides power to any or all components of circuit board 150 (e.g., CPU 152, display 153, storage device 158, sensor 160, etc., or combinations thereof).

For example, energy harvesting device 154 may be a thermoelectric or Seebeck generator 154a configured to convert thermal energy into electrical energy. Thermoelectric generator 154a may include P-Type and N-Type semiconductors disposed between two dissimilar materials, e.g., metals, ceramics, substrates, or the like. In use, as surgical instrument 100 receives a concentration of thermal energy from the sterilization device, a temperature differential is created between a hot side of thermoelectric generator 154a on an outer surface of surgical instrument 100 that is exposed directly to the heat generated by the sterilization device, and a cool side of thermoelectric generator 154a in/on an inner surface of the surgical instrument 100. Thermoelectric generator 154a converts the thermal energy into electrical energy, or voltage, as a result of the temperature differential between each side of the thermoelectric generator 154a, and/or between the respective inner and outer surfaces of the surgical instrument 100.

In some embodiments, implementation may be effectuated via a piezo-electric device such as a Peltier. For instance, rather than being used as an output transducer, the piezo-electric device is configured to determine a difference in temperature on the sides of the device to generate an electric potential.

In certain embodiments, the voltage can be approximated using the following formula, V=(S_{B}−S_{A})×(T₂−T₁), where V is the generated voltage, S_{A} and S_{B} are the respective Seebeck coefficients of the dissimilar materials used for thermoelectric generator 154a, and T₁ and T₂ are the respective temperatures of the relatively hot and cold sides of the thermoelectric generator 154a and/or the outer and inner surfaces of surgical instrument 100. The voltage created by the thermoelectric generator 154a can be used to power the circuit board 150 and surgical instrument 100 for performing certain functions during a sterilization procedure in a sterilization device, as will be described herein below.

Additionally or alternatively, energy harvesting device 154 may include a photovoltaic or solar cell 154b configured to convert light energy into electrical energy. For example, when surgical instrument 100 is placed in a sterilization device that uses a light source to sterilize surgical instrument 100, solar cell 154b of harvesting device 154 may be used to convert the light energy from the sterilization device into electrical energy to provide power to surgical instrument 100. In embodiments, solar cell 154b may be formed from crystalline silicon, thin film, multijunction cells, or the like.

Additionally or alternatively, energy harvesting device 154 may include a piezoelectric transducer 154c (e.g., a Peltier) configured to convert kinetic energy (e.g., pressure, vibrations, movements, waves, sounds, temperature, etc., or combinations thereof) produced by a sterilization device into electrical energy to provide power to surgical instrument 100. Piezoelectric transducer 154c may be formed from any suitable material including quartz, berlinite, sucrose, rochelle salt, topaz, tourmaline-group minerals, lead titanate, silk, wood, synthetic crystals, synthetic ceramics, lead-free piezoceramics, III-V and II-VI semiconductors, polymers, organic nanostructures, or the like.

An amplifier 156 of circuit board 150 may be configured to operate in conjunction or may be operatively (e.g., directly and/or indirectly) connected to energy harvesting device 154 to condition the voltage generated by energy harvesting device 154. Amplifier 156 may be any type of amplifier, such as a transistor, vacuum-tube, magnetic, negative resistance, circuit, operational, differential, switched mode, etc., or combinations thereof.

In accordance with the present disclosure, the energy that is harvested is in Joules of energy, and the potential of this energy is measured in volts. The voltage is typically too low to power electronics. Accordingly, the voltage needs to be conditioned by an amplifier to amplify the voltage output. Specifically, for example, the energy harvester outputs the energy at 1V and the electronics need 3V to operate. This would typically be done using a DC/DC converter, which could be considered a type of amplifier because it is boosting the voltage while the amount of energy stays the same. The output of this “amplifier” section is then at a higher potential (voltage) and powers the electronics. The amount of power used is determined by the voltage output of the “amplifier” multiplied by the current consumed by the electronics.

An energy storage device 158 of circuit board 150 may be configured to store the energy generated from energy harvesting device 154 to provide power to surgical instrument 100, or components thereof. The energy storage device 158 may allow CPU 152 to operate for an extended period of time, e.g., to record data during an entire sterilization cycle in a sterilization device. Energy storage device 158 may also be configured to provide a burst of power when energy is desired or required more quickly such as if this were to be used to permanently disable the device (e.g., self-destruction). The energy storage device 158 may be any suitable device configured to store energy, such as, e.g., a capacitor, a battery, or the like.

Sensor 160 of surgical instrument 100 is operatively (e.g., directly and/or indirectly) connected to CPU 152 and may be configured to measure the energy output of a sterilization device, as applied to surgical instrument 100. The threshold energy output required to sufficiently sterilize surgical instrument 100 (e.g., remove harmful bacteria or other infectious agents from surgical instrument 100) may be based on empirical or experimental data, which may be programmed or pre-programmed into CPU 152 of circuit board 150. Once the threshold energy output is met or measured by sensor 160, CPU 152 will count a full sterilization cycle and record the sterilization data into memory 152a of CPU 152.

For example, sensor 160 may be a temperature sensor configured to measure temperature of surfaces of the surgical instrument 100. If an outer surface of surgical instrument 100 reaches a threshold temperature, e.g., a temperature sufficient for sterilization of surgical instrument 100, and maintains the threshold temperature for a predetermined period of time, then CPU 152 will record a full sterilization cycle and record the data in memory 152a.

Sensor 160 can be configured to determine and calculate other forms of energy output applied to surgical instrument 100 sufficient to sterilization surgical instrument 100, such as a light energy, kinetic energy, or the like. As described above, once a threshold energy output is measured by sensor 160, CPU 152 records a full sterilization cycle, which is recorded into memory 152a.

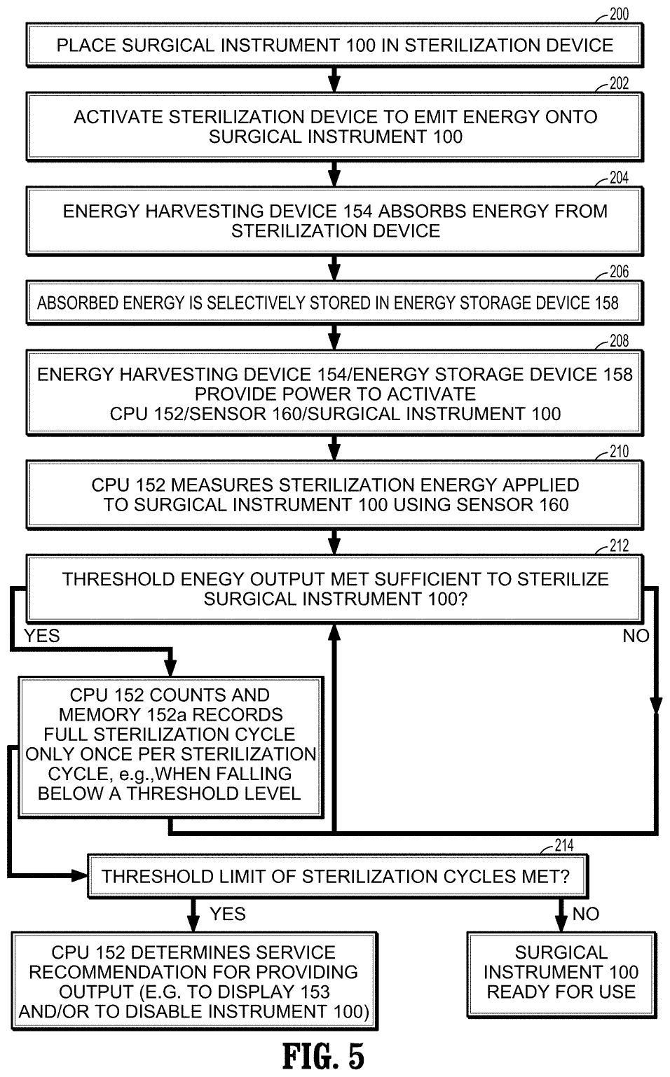

With reference to FIG. 5, a flowchart depicting operation of surgical instrument 100 during a sterilization procedure is provided. In step 200, surgical instrument 100 is placed in a sterilization device (e.g., an autoclave, pressure chamber, or the like, not explicitly shown) to undergo sterilization. In step 202, the sterilization device is activated such that energy (e.g., thermal, light, pressure, kinetic, etc., or combinations thereof) is applied to surgical instrument 100 to sterilize surgical instrument 100. In step 204, the energy harvesting device 154 harvests the energy from the sterilization device. The energy harvested from energy harvesting device 154 may be conditioned (e.g., voltage boosted) by amplifier 156.

In step 206, energy storage device 158 may selectively store the absorbed energy from energy harvesting device 154. In some aspects, if the power requirements of circuit board 150 are minimal, energy storage device 158 may be powered without step 206 (e.g., directly from the energy harvesting element). In certain aspects, this may also apply to the amplifier/boost/condition circuit. In step 208, energy harvesting device 154 and/or the energy storage device 158 provide power to CPU 152 such that CPU 152, sensor 160, or combinations thereof are “powered on” and can begin performing functions. In step 210, sensor 160 measures the energy output of the sterilization device, as applied to the surgical instrument 100. CPU 152 then determines if a threshold energy output (e.g., a maximum temperature, light, kinetic, pressure, or energy value) sufficient for sterilization of surgical instrument 100 has been met. In step 210, if the threshold energy output is met, CPU 152 counts and memory 152a records a full or completed sterilization cycle. In accordance with the present disclosure, the usage count is only changed once per sterilization cycle (e.g., the measured applied energy must fall below a certain predetermined threshold before the usage count is changed again). Once the sterilization cycle is complete, in step 214, CPU 152 determines if surgical instrument 100, or any parts thereof, has undergone a threshold limit of sterilization cycles. CPU 152 then determines and optionally display 153 displays a service recommendation. Additionally or alternatively, a user can download the data onto an external device (e.g., tablet, smartphone, computer, etc., or combinations thereof) using data interface device 153a. If the threshold limit of sterilization cycles is not met, then the surgical instrument 100 is ready for use. It is contemplated that the data (e.g., usage count) may also be read by the instrument drive unit 50 through a data interface, or, alternatively, surgical instrument 100 may be disabled.

Although surgical instrument 100 is configured for use with robotic surgical systems, it should be appreciated that the disclosed systems and methods are applicable to any type of surgical instrument, device, tool, or assembly, such as for example, the powered handheld surgical instruments described in commonly owned U.S. Pat. Nos. 8,968,276 and 9,055,943, and commonly owned U.S. Patent Application No. 2016/0310134, the entire contents of each of which is hereby incorporated by reference.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

What is claimed is:

1. A surgical device, comprising:
an energy harvesting device configured to harvest energy from a sterilization device to provide power to at least one of:
a sensor configured to measure an energy output applied to the surgical device by a sterilization device; or
a computing device including a processor or logic circuit and a memory, the computing device configured to:
determine when a threshold energy output applied to the surgical device is met; and
store in the memory an amount of times the threshold energy output applied to the surgical device is met.

2. The surgical device of claim 1, wherein when the threshold energy output applied to the surgical device is met, the computing device counts and the memory records a completed sterilization cycle.

3. The surgical device of claim 2, wherein the computing device is configured to determine when the surgical device requires servicing based on a number of completed sterilization cycles.

4. The surgical device of claim 1, wherein the energy harvesting device is a thermoelectric generator configured to receive a concentration of thermal energy from a sterilization device, the thermoelectric generator configured to convert a concentration of thermal energy into electrical energy to power the surgical device.

5. The surgical device of claim 2, wherein the sensor is a temperature sensor configured to measure a temperature of an outer surface of the surgical device.

6. The surgical device of claim 5, wherein the temperature sensor is operatively coupled to the computing device, the computing device configured to detect a threshold temperature necessary to sterilize the surgical device.

7. The surgical device of claim 6, wherein when the computing device detects a threshold temperature, the computing device counts and the memory records a completed sterilization cycle.

8. The surgical device of claim 1, wherein the surgical device includes a display configured for displaying at least one output of the computing device.

9. The surgical device of claim 1, further comprising an energy storage device configured to store the harvested energy from the energy harvesting device.

10. The surgical device of claim 9, wherein the energy storage device is a capacitor.

11. The surgical device of claim 1, further comprising an amplifier operatively connected to the energy harvesting device, the amplifier configured to condition the power of the harvested energy from the energy harvesting device.

12. The surgical device of claim 1, wherein the energy harvesting device includes a photovoltaic cell configured to convert light energy from a sterilization device into electrical energy to power the surgical device.

13. The surgical device of claim 1, wherein the energy harvesting device includes a piezoelectric transducer configured to convert kinetic energy into electrical energy to power the surgical device.

14. The surgical device of claim 1, further comprising a data interface device configured to interface with an external device, the data interface device configured to upload data from the computing device onto the external device, the data interface device configured to download data from the external device onto the computing device.

15. A method for operation of a surgical instrument during sterilization, comprising:
placing a surgical instrument in a sterilization device, the surgical instrument including:
an energy harvesting device configured to harvest energy from a sterilization device;
a sensor configured to measure an energy output applied to the surgical device by a sterilization device; and
a computing device including a processor or logic circuit and a memory;
powering the sensor and the computing device using the harvested energy from the energy harvesting device;
determining, with the computing device and the sensor, when a threshold energy output applied to the surgical device is met; and
storing in the memory an amount of times that the threshold energy output applied to the surgical device is met.

16. The method of claim 15, further comprising counting, with the computing device, a completed sterilization cycle when a threshold energy output is applied to the surgical device.

17. The method of claim 15, further comprising disabling the surgical device with the computing device when a threshold number of completed sterilization cycles is met.

18. The method of claim 15, further comprising storing the harvested energy from the energy harvesting device with an energy storage device.

19. The method of claim 15, further comprising displaying on a display device at least one output of the computing device.

20. The method of claim 15, further comprising conditioning the harvested energy from the energy harvesting device with an amplifier.
